(19) United States
(12) Patent Application Publication
(10) Pub. No.: US 2008/0234258 A1
(43) Pub. Date: Sep. 25, 2008

(54) Dihydroxyanthraquinones and Their Use

(76) Inventors: Andrew Douglas Baxter, Cambridge (GB); Andrea Walmsley, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/997,660

(22) PCT Filed: Aug. 10, 2006
(86) PCT No.: PCT/GB06/02999

§ 371 Date: May 20, 2008

(30) Foreign Application Priority Data
Aug. 10, 2005 (GB) 0516469.4

(51) Int. Cl. A61K 31/5377; C07D 413/14; C07D 407/12; A61K 31/351; A61P 35/04; A61P 1/00; A61P 17/00; A61P 3/00

(52) U.S. Cl. 514/231.5; 544/149; 549/415; 514/451

(57) ABSTRACT

A compound of formula (1)

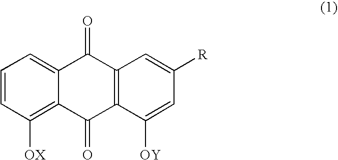

wherein X is H or —OCR₁ and Y is H or —OCR₂, provided that X and Y are not both H; and R is CH₂OR₉, CONR₁₁R₁₂, CN, tetrazole or COOR₁₇; or a salt thereof, has therapeutic utility.

### FIELD OF THE INVENTION

The present invention relates to dihydroxyanthraquinone derivatives and to their use in the treatment of disease.

### BACKGROUND TO THE INVENTION

T-lymphocytes are known to play a central role in the pathogenesis of many inflammatory and autoimmune diseases. The activation of T-cells by antigen-presenting cells is the primary event in the initiation of the inflammatory process, which subsequently leads to the activation of other inflammatory cells and in turn the release of pro-inflammatory cytokines, chemotactic agents and matrix degrading enzymes. It is also known that these cell types are essential for the initiation and maintenance of angiogenesis through the secretion of VEGF. Angiogenesis-driven diseases include but are not limited to cancer, age-related macular degeneration and diabetic retinopathy.

Multiple sclerosis is a chronic demyelinating inflammatory disease of the central nervous system. T-cell proliferation leads to release of the pro-inflammatory cytokines (primarily IL-2 and IFN-γ) and the recruitment of leucocytes (including macrophages) which orchestrate the inflammatory response.

In chronic obstructive pulmonary disease (COPD), activation of neutrophils and macrophages by proliferating CD8+ T-cells leads to the release of pro-inflammatory cytokines and elastin-degrading enzymes such as neutrophil elastase (HNE) and metalloelastase (MMP-12), which causes a chronic and progressive degradation of lung tissues and ultimately reduction in respiratory function.

Crohn's disease and ulcerative colitis are chronic inflammatory diseases of the intestines collectively known as inflammatory bowel disease (IBD). It is likely that both these disorders are actually heterogeneous groups of diseases that have different causes, but share similar perpetuating stimuli and common pathways of tissue injury. Once again, T-cells are central to the progression of this collection of diseases, leading to the activation of immune, mesenchymal and epithelial cells, recruitment of circulating effector cells and ultimately gastrointestinal tissue damage.

In psoriasis, the presentation of antigen by Langerhan's cells to CD4+ T-cells leads to the synthesis of cytokines which stimulate keratinocyte proliferation and the expression of adhesion molecules by endothelial cells and keratinocytes. Keratinocytes in turn are stimulated to produce their own cytokines which can act in an autocrine and/or paracrine manner to maintain the psoriatic process.

There is a similarly strong rationale for the central involvement of T-cells in many other inflammatory diseases, including systemic lupus erythematosus (SLE), lupus nephritis, glomerulonephritis, IgA nephropathy, periodontal disease, atopic dermatitis, scleroderma, graft vs host disease allopeicia, Sjogren's syndrome, polymyosititis, pempligus, uveititis and Addison's disease. Thus, inhibitors of T-cell proliferation are much sought after and may have utility in the treatment of the range of inflammatory and autoimmune diseases described in detail above.

Rhein (1,8-dihydroxyanthraquinone-3-carboxylic acid) is a well characterised anti-inflammatory agent with recognised utility in a range of inflammatory diseases. While this agent has not been demonstrated to inhibit T-cell proliferation, it is known to inhibit the production of pro-inflammatory cytokines (IL-1β and TNFα) in human osteoarthritic synovium and chondrocytes (Martel-Pelletier et al, Journal of Rheumatology, 1998, 25 (4), 753-762) and to inhibit cytokine gene expression in a model of lupus nephritis (Lemay et al, Kidney International, 1996, 50 (1), 85-93). In common with the tetracyclines, rhein and its pro-drug diacerein have been shown to down-regulate the production of pro-matrix metalloproteinases (pro-MMPs-1, -3, -9 and -13), while upregulating the production of tissue inhibitor of metalloproteinases (TIMP-1) from rabbit articular chondrocytes (Tamura et al, Osteoarthritis and Cartilage, 2001, 9 (3), 257-263).

U.S. Pat. No. 4,346,103 discloses the use of rhein in arthritis and multiple sclerosis. Its use in diabetic nephropathy is disclosed in EP0990441. In these diseases, over-production of IL-1β is particularly implicated. The more widespread use of rhein has been somewhat limited by its rather poor physicochemical properties. This issue is not addressed completely with the well characterised pro-drug diacerein, where utility in the clinical setting is again limited by poor physicochemical properties (P. Nicolas et al, Clin. Pharmacokinet., 1998, 35 (5), 347-359).

### SUMMARY OF THE INVENTION

The present invention is related to an observation that simple derivatives of rhein are capable of inhibiting cytokine production and T-cell proliferation in assays where rhein itself and other simple derivatives fail to produce a response. It is likely that these agents will be of clinical utility in the wide range of inflammatory and autoimmune diseases described above, due to their improved physical properties over the parent compound.

According to the present invention, novel compounds are of formula (1)

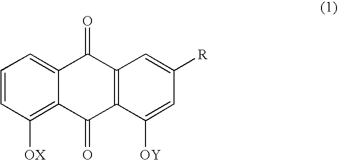

wherein X is H or —OCR₁ and Y is H or —OCR₂, provided that X and Y are not both H; and

R is CH₂OR₉, CONR₁₁R₁₂, CN, tetrazole or COOR₁₇; wherein

R₁ and R₂ are independently selected from C₁₋₄ alkyl substituted with R₃, and a four to seven-member cyclic radical optionally substituted with a group selected from CF₃, OR₄, NR₅R₆, S(O)₀₋₂R₇, C₁₋₄ alkyl optionally substituted with R₃, and halogen, the radical also optionally containing one or more heteroatoms selected from O, S(O)₀₋₂ and NR₉;

R₃ is CF₃, OR₄, NR₅R₆ or S(O)₀₋₂R₇; or

R₄, R₅ and R₆ are independently C₁₋₄ alkyl optionally substituted with R₃, or NR₅R₆ is a C₄₋₆ heterocycloalkyl ring containing one or more additional heteroatoms selected from O, NR₉ and S(O)₀₋₂;

R₇ is C₁₋₄ alkyl;

R₉ is H or C₁₋₄ alkyl;

R₁₁ and R₁₂ are independently selected from H, OH, C₁₋₄alkyl optionally substituted with R₁₃, C₃₋₆ cycloalkyl optionally substituted with R₁₄, and C₄₋₆ heterocycloalkyl optionally substituted with R₁₄; or NR₁₁R₁₂ is a four to seven-member cyclic radical optionally substituted with R₁₄ and optionally containing one or more heteroatoms selected from O, S(O)₀₋₂ and NR₁₅;

R₁₃ is aryl optionally substituted with R₁₄, heteroaryl optionally substituted with R₁₄, C₃₋₆ cycloalkyl optionally substituted with R₁₄, or C₄₋₆ heterocycloalkyl optionally substituted with R₁₄;

R₁₄ is OR₉, CO₂R₉ or N(R₉)₂, where each R₉ is as defined above, or N(R₉)₂ is a four to seven-member cyclic radical optionally containing one or more additional heteroatoms selected from O, S(O)₀₋₂ and NR₁₅; or N(R₉)₂ is a 5 or 6-member cyclic radical such as a lactam, succinimide or hydantoin of the formula

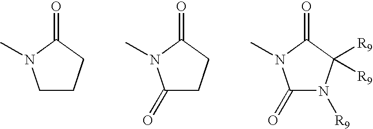

R₁₅ is R₉ or COR₁₆;

R₁₆ is C₁₋₄ alkyl, aryl or heteroaryl; and

R₁₇ is C₁₋₄ alkyl optionally substituted with R₁₃, C₃₋₆ cycloalkyl optionally substituted with R₁₄, or C₄₋₆ heterocycloalkyl optionally substituted with R₁₄;

or a salt thereof.

According to a further aspect of the invention, a compound of formula (1) is used for the manufacture of a medicament for therapy of a condition associated with T-cell proliferation or that is mediated by pro-inflammatory cytokines, particularly IL-1β or IL-18.

Thus, an effective amount of a compound of formula (1) can be used in a method for the treatment of such a condition, in a patient in need thereof.

### DESCRIPTION OF PREFERRED EMBODIMENTS

It will be appreciated that compounds according to the invention can contain one or more asymmetrically substituted carbon atoms. The presence of one or more of these asymmetric centres in a compound of formula (1) can give rise to stereoisomers, and in each case the invention is to be understood to extend to all such stereoisomers, including enantiomers and diastereomers, and mixtures including racemic mixtures thereof.

The term “C₁₋₄ alkyl” refers to a straight or branched chain alkyl moiety having from one to four carbon atoms, including for example, methyl, ethyl, propyl, isopropyl, butyl, tert-butyl and the like.

The term “C₄₋₆ heterocycloalkyl” refers to a saturated heterocyclic moiety having from three to six carbon atoms and one or more heteroatoms selected from N, O and S, and includes for example azetidinyl, oxetidinyl, pyrrolidinyl, tetrahydrofuranyl, piperidinyl, tetrahydropyranyl and the like.

The term “four to seven-member cyclic radical” refers to a saturated or unsaturated carbocyclic or heterocyclic moiety having four to seven ring atoms which may include one or more heteroatoms (as defined above).

“Aryl” and “heteroaryl” should be interpreted analogously. Each may have one or two fused rings, and contain up to 10 ring atoms. Examples include phenyl, naphthyl, furyl, pyridyl and thiophenyl.

The term “halogen” means fluorine, chlorine, bromine or iodine.

A carboxyl group can be protected in the form of a readily cleavable ester such as the methyl, ethyl, benzyl or tert-butyl ester.

Compounds of the invention may be in the form of a hydrate or solvate.

Salts of compounds of formula (1) include pharmaceutically acceptable salts, for example acid addition salts derived from inorganic or organic acids, such as hydrochlorides, hydrobromides, p-toluenesulphonates, phosphates, sulphates, perchlorates, acetates, trifluoroacetates, propionates, citrates, malonates, succinates, lactates, oxalates, tartrates and benzoates.

Salts may also be formed with bases. Such salts include salts derived from inorganic or organic bases, for example alkali metal salts such as magnesium or calcium salts, and organic amine salts such as morpholine, piperidine, dimethylamine or diethylamine salts.

Compounds of formula (1) may be prepared by any suitable method known in the art and/or by the process described below. It will be appreciated that where a particular stereoisomer of formula (1) is required, the synthetic processes described herein may be used with the appropriate homochiral starting material and/or isomers maybe resolved from mixtures using conventional separation techniques (e.g. HPLC).

It will also be appreciated that functional groups, such as amino, hydroxyl or carboxyl groups, present in the various compounds described below, and which it is desired to retain, may need to be in protected form before any reaction is initiated. In such instances, removal of the protecting group may be the final step in a particular reaction. Suitable protecting groups for such functionality will be apparent to those skilled in the art. For specific details see “Protective Groups in Organic Synthesis”, Wiley Interscience, T W Greene, P G M Wuts.

A process for preparing compounds of general formula (1) wherein X is —OCR₁ and Y is —OCR₂ comprises conversion of the activated ester in the presence of base (such as diacerein to rhein), followed by reaction with the required activated acid such as acid chloride or anhydride. The carboxylic acid can be reduced to give the alcohol and the hydroxyl group further substituted, or desired amides and esters can be formed by reacting the carboxylic acid or acid chloride with suitable amines, or alcohols or alkyl halides, respectively. Diacerein and the corresponding activated acids are either commercially available or readily obtained from commercially available materials by people who are skilled in the art of synthetic organic chemistry.

In order to prepare a compound of formula (1) wherein X or Y is hydrogen, the process will be similar to that described above, but will necessitate the additional steps of selectively protecting one hydroxyl group prior to the reaction with the activated acid, and this will have to be followed by a deprotection step to reveal the target compound.

Any mixtures of final products or intermediates obtained can be separated on the basis of the physico-chemical differences of the constituents, in known manner, into the pure final products or intermediates, for example by chromatography, distillation, fractional crystallization, or by formation of a salt if appropriate or possible under the circumstances.

Compounds of formula (1) according to the invention exhibit in vitro inhibiting activities with respect to T-cell proliferation. Compounds according to the invention also exhibit in vitro inhibition of pro-inflammatory cytokine release. The activity of the compounds may be determined by use of the appropriate cellular assay, for example as described below.

This invention also relates to a method of treatment for patients (including man and/or mammalian animals raised in the dairy, meat or fur industries or as pets) suffering from disorders or diseases which can be attributed to T-cell proliferation as previously described, and more specifically, a method of treatment involving the administration of the T-cell proliferation inhibitors of formula (1) as the active constituents.

Accordingly, the compounds of formula (1) can be used among other things in the treatment of osteoarthritis and rheumatoid arthritis, psoriasis, systemic lupus erythromatosis (SLE), multiple sclerosis, chronic obstructive pulmonary disease (COPD) and inflammatory bowel disease including ulcerative colitis, Crohn's disease, cancer, diabetic retinopathy and age-related macular degeneration.

As mentioned above, compounds of formula (1) are useful in human or veterinary medicine since they are active as inhibitors of T-cell proliferation. Accordingly in another aspect, this invention concerns a method of management (by which is meant treatment or prophylaxis) of disease or conditions mediated by T-cells in mammals, in particular in humans, which method comprises administering to the mammal an effective amount of a compound of formula (1) above, or a pharmaceutically acceptable salt thereof; a compound of formula (1) for use in human or veterinary medicine, particularly in the management (by which is meant treatment or prophylaxis) of diseases or conditions mediated by T-cells; and the use of a compound of formula (1) in the preparation of an agent for the management (by which is meant treatment or prophylaxis) of diseases or conditions mediated by T-cells.

The disease or conditions referred to above include inflammatory and autoimmune diseases such as rheumatoid arthritis, osteoarthritis, osteoporosis, Crohn's disease, ulcerative colitis, multiple sclerosis, periodontitis, gingivitis, graft versus host reactions, psoriasis, scleroderma, allopeicia, Sjogren's syndrome, polymyosititis, pempligus, uveititis, Addison's disease, atopic dermatitis, asthma, systemic lupus erythematosus (SLE), nephropathy and chronic obstructive pulmonary disease (COPD), cancer, diabetic retinopathy and age-related macular degeneration.

For the treatment of rheumatoid arthritis, multiple sclerosis, and in other diseases and indications resulting from the over-activity of T-cells such as those highlighted above, compounds of formula (1) may be administered orally, topically, parenterally, by inhalation or nasal spray or rectally in dosage unit formulations containing non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats etc, the compounds of the invention are effective in the treatment of humans.

A pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from sweetening agents, flavouring agents, colouring agents and preserving agents, in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastointestinal tract and thereby provide a sustained action over a longer period. For example, a time delay material such as glyceryl monostearate or glyeryl distearate may be employed. They may also be coated by the techniques described in U.S. Pat. No. 4,256,108, U.S. Pat. No. 4,166,452 and U.S. Pat. No. 4,265,874, to form osmotic therapeutic tablets for control release.

Formulations for oral use may also be presented as hard gelatin capsules where in the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavouring and colouring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example gycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

A compound of formula (1) may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, containing a compound of formula (1) may be employed. For purposes of this Application, topical application includes mouth washes and gargles.

Dosage levels of the order of from about 0.05 mg to about 140 mg per kilogram of body weight per day are useful in the treatment of the above-indicated conditions (about 2.5 mg to about 7 g per patient per day). For example, inflammation may be effectively treated by the administration of from about 0.01 to 50 mg of the compound per kilogram of body weight per day (about 0.5 mg to about 3.5 g per patient per day).

The amount of active ingredient that may be combined with the carrier materials to produce a single dosage form will vary depending upon the host treated and the particular mode of administration. For example, a formulation intended for the oral administration of humans may vary from about 5 to about 95 percent of the total composition. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

The following Examples illustrate the invention. The starting material for Examples 1, 2 and 4 (4,5-ditetrahydropyranoyloxy-9,10-dioxo-9,10-dihydroanthracene-2-carboxylic acid) was prepared as described in WO2005/085170.

### Example 1

### 1,8-bis(tetrahydropyran-4-carbonyloxy)-3-(morpholine-4-carbonyl)-9,10-dioxo-9,10-dihydroanthracene

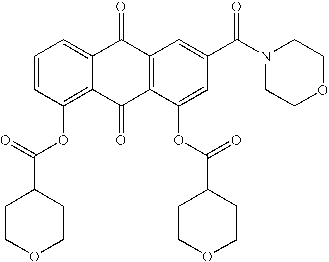

To a 100 mL flask equipped with magnetic stirrer, 4,5-ditetrahydropyranoyloxy-9,10-dioxo-9,10-dihydroanthracene-2-carboxylic acid (2.00 g, 3.9×10⁻³ mol) was stirred in DCM (50 mL) under nitrogen. EDCI (0.90 g, 4.7×10⁻³ mol) and HOBt (0.65 g, 4.7×10⁻³ mol) were added and the solution was allowed to stir for 15 min. The solid suspension dissolved into the DCM solvent. Morpholine (0.54 mL, 5.9×10⁻³ mol) was then added and the solution was allowed to stir for 30 minutes.

The reaction mixture was diluted in 300 mL of DCM. The organic layer was washed with 200 mL of saturated NaHCO₃ solution, and 200 mL of saturated NaCl solution. The organic layer was separated and evaporated to dryness under reduced pressure. The resulting residue was then triturated in water, filtered, and dried under high-reduced pressure, yielding the desired product as a yellow solid (1.95 g, 87%).

¹H NMR (500 MHz, DMSO) δ=8.1 (d, 1H, Ar), 8.0 (s, 1H, Ar), 7.9 (t, 1H, Ar), 7.7 (s, 1H, Ar), 7.6 (d, 1H, Ar), 3.9 (d, 4H, THP group), 3.6 (broad, 4H, morpholine), 3.5 (broad, 2H, morpholine), 3.4 (t, 4H, THP group), 3.0 (m, 2H, morpholine), 2.5 (s, 2H, THP group), 2.0 (d, 4H, THP group), 1.8 (q, 4H, THP group).

### Example 2

### 1,8-bis(tetrahydropyran-4-carbonyloxy)-3-ethylcarbamoyl-9,10-dioxo-9,10-dihydroanthracene

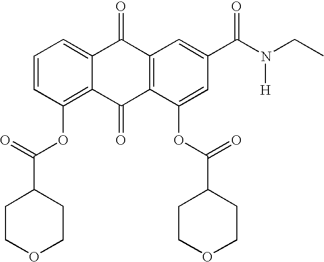

To a 100 mL flask equipped with magnetic stirrer, 4,5-ditetrahydropyranoyloxy-9,10-dioxo-9,10-dihydroanthracene-2-carboxylic acid (2.00 g, 3.9×10⁻³ mol) was stirred in DCM (50 mL) under nitrogen. EDCI (0.90 g, 4.7×10³ mol) and HOBt (0.65 g, 4.7×10³ mol) were added and the solution was allowed to stir for 15 min. The solid suspension dissolved into the DCM solvent. Ethylamine (3.00 mL, 5.9×10⁻³ mol) was then added and the solution was allowed to stir for 15 minutes.

The reaction mixture was then diluted in 300 mL of DCM. The organic layer was washed with 200 mL of saturated NaHCO₃ solution, and 200 mL of saturated NaCl solution. The organic layer was separated and evaporated to dryness under reduced pressure. The resulting residue was triturated in water, filtrated, and dried under high-reduced pressure, yielding the desired product as a yellow solid (1.56 g, 75%).

¹H NMR (500 MHz, DMSO) consistent with structure

δ=9.0 (s, 1H, N—H), 8.5 (t, 1H, Ar), 8.1 (d, 1H, Ar), 8.0 (s, 1H, Ar), 7.9 (t, 1H, Ar), 7.6 (d, 1H, Ar), 3.9 (d, 4H, THP group), 3.4 (t, 4H, THP group), 2.9 (q, 2H, CH₂), 2.5 (s, 2H, THP group), 2.0 (d, 4H, THP group), 1.8 (q, 4H, THP group), 1.2 (t, 3H, CH₃).

### Example 3

### 4,5-bis(tetrahydropyran-4-carbonyloxy)-9,10-dioxo-9,10-dihydroanthracene-2-carboxylic acid, 2-methoxyethyl ester

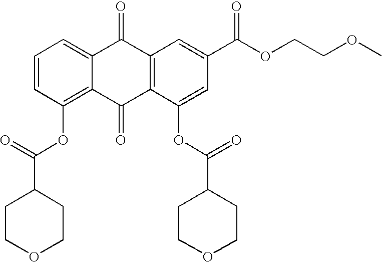

To a 250 mL flask equipped with magnetic stirrer, Dean-Stark apparatus and under nitrogen was charged 2-methoxyethanol (300 mL) and 4,5-dihydroxy-9,10-dioxoanthracene-2-carboxylic acid (3.00 g, 10.6×10⁻³ mol) with 0.1 mL of H₂SO₄. The mixture was refluxed for 24 hours, (external oil bath temperature 145° C.). The reaction mixture was then allowed to cool at RT.

The resulting solid was filtrated and washed with 10 mL of chilled Et₂O. 4,5-dihydroxy-9,10-dioxoanthracene-2-carboxylic acid 2-methoxyethyl ester was isolated as a yellow solid 2.9 g, 81%).

¹HNMR analysis was consistent with the desired product, (500 MHz, DMSO) δ=8.1 (s, 1H, Ar), 7.8 (t, 1H, Ar), 7.7 (s, 1H, Ar), 7.7 (d, 1H, Ar), 7.4 (d, 1H, Ar), 4.4 (t, 2H, COOCH₂), 3.7 (t, 2H, CH₂OCH₃), 3.3 (s, 3H, CH₂OCH₃).

To a 250 mL flask equipped with magnetic stirrer, reflux condenser, pressure equalizing dropping funnel, and under nitrogen, was added 4,5-dihydroxy-9,10-dioxoanthracene-2-carboxylic acid 2-methoxyethyl ester (2.70 g, 7.88×10⁻³ mol), followed by pyridine (85 mL). When all starting material was dissolved, tetrahydropyran-4-carboxylic acid chloride (4.53 g, 31.52×10⁻³ mol) was added dropwise and the solution was stirred at room temperature for 48 hours. TLC analysis 80:20 (DCM:MeOH) indicated no starting material present.

Water (250 mL) was added and extracted with ethyl acetate (4×400 mL). The organic layers were combined, washed with water (2×300 mL) and saturated NaCl solution (2×300 mL). The organic layer was dried over anhydrous MgSO₄, filtered, and evaporated to dryness under reduced pressure. The resulting solid was then triturated in Et₂O, filtered, and dried under reduced pressure to give 4,5-bis(tetrahydropyran-4-carbonyloxy)-9,10-dioxo-9,10-dihydroanthracene-2-carboxylic acid, 2-methoxyethyl ester as a yellow solid (3.17 g, 71%).

¹H NMR consistent with structure (500 MHz, DMSO) δ=8.8 (s, 1H, Ar), 8.2 (d, 1H, Ar), 8.0 (s, 1H, Ar), 7.7 (t, 1H, Ar), 7.3 (d, 1H, Ar), 4.5 (t, 2H, COOCH₂), 4.0 (t, 4H, THP group), 3.7 (t, 2H, CH₂OCH₃), 3.5 (m, 4H, THP group), 3.4 (s, 3H, CH₂OCH₃), 2.9 (m, 2H, THP group), 2.1 (m, 4H, THP group), 2.0 (m, 4H, THP group).

### Example 4

### 4,5-bis(tetrahydropyran-4-carbonyloxy)-9,10-dioxo-9,10-dihydroanthracene-2-carboxylic acid, ethyl ester

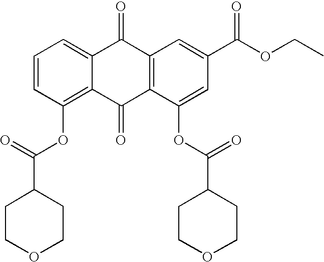

4,5-Ditetrahydropyranoyloxy-9,10-dioxo-9,10-dihydroanthracene-2-carboxylic acid (5 g, 9.8 mmol) was dissolved in DMF (200 ml) and sodium hydride (432 mg, 10.8 mmol) was added at room temperature. The resulting yellow precipitate was stirred for 30 min at room temperature, and then ethyl iodide (0.94 ml, 11.8 mmol) was added. The mixture was stirred at 50° C. for 48 h and was poured onto water (300 ml). The aqueous was extracted with ethyl acetate (3×200 ml) and the organics were dried (MgSO₄) and evaporated to dryness to give a brown solid. This was triturated with diethyl ether (40 ml) to give an orange solid (2.4 g, 45%).

¹H NMR (400 MHz, DMSO): 1.38 (3H, t J=7.2 Hz), 1.76-1.89 (4H, m), 2.00-2.04 (4H, m), 2.95-2.99 (2H, m), 3.45 (4H, t J=11.2 Hz), 3.94-3.96 (4H, m), 4.42 (2H, q J=7.2 Hz), 7.65 (1H, dd J=8.4, 1.2 Hz), 7.96 (1H, t J=8.4 Hz), 8.08 (1H, d J=2 Hz), 8.15 (1H, dd J=8.4, 1.2 Hz), 8.54 (1H, d J=2 Hz). ¹³C NMR (100 MHz, DMSO): 14.62, 28.75, 28.82, 62.58, 66.71, 125.38, 125.70, 126.02, 128.91, 130.73, 131.35, 134.66, 135.22, 135.72, 136.06, 150.11, 150.39, 164.11, 172.69, 172.75, 180.71, 181.29; LC-MS: 537 (M+H⁺).

The following compounds were obtained in analogous manner.

### Example 5

### 4a,9,9a,10-Tetrahydro-4,5-bis(2-(tetrahydro-2H-pyran-4-yl)-2-oxoethyl)-9,10-dioxoanthracene-2-nitrile

### Example 6

### 4a,9,9a,10-Tetrahydro-4,5-bis(2-(tetrahydro-2H-pyran-4-yl)-2-oxoethyl)-9,10-dioxoanthracene-2-(1H-tetrazol-5-yl)

### LPS Mouse Assay

7 week old Balb C ByJ mice (24-28 g) were administered, either by oral (10 ml/kg) administration, with vehicle or test article. 30 minutes later, these animals were challenged with an intraperitoneal injection of 1 mg/kg LPS. 2 hours after LPS challenge, blood samples were collected under light isoflurane anaesthesia into normal tubes by retro-orbital puncture. Samples were allowed to clot at room temperature and then spun at 6000 g for 3 min at 4° C. Serum was stored at −20° C. until use. Serum TNFα and IL-10 levels were analysed in duplicate by ELISA technique.

30 mg/kg of the compound of Example 1 produced an effect on LPS-induced TNFα secretion in mice, while 3, 10 and 30 mg/kg reduced IL-1β levels from the same LPS stimulus. This demonstrates a multi-cytokine effect on the innate stimulated immune system, primarily through an effect on IL-1β.

10 mg/kg of the compound of Example 2 inhibited LPS-induced TNFα secretion. This demonstrates a TNFα effect on the innate stimulated immune system.

30 mg/kg dose of the compound of Example 3 produced an effect on LPS-induced TNFα secretion. This demonstrates a TNFα effect molecule on the innate stimulated immune system.

### Carrageenan Paw Assay

Fasted (18 hour) male Wistar rats (105-130 g) were weighed and a basal mercury plethysmometer reading was taken of the right hind paw by submerging the paw in the mercury up to the tibiotarsal joint. Subsequently, vehicles, reference items and test articles were administered by oral gavage (10 ml/kg). 30 minutes after treatment, 0.1 ml of 2% carrageenan in 0.9% saline was injected into the subplanatar area of the right hind paw. The right paw was measured again with the plethysmometer, at 1, 2, 3, 4 and 5 hours after carrageenan administration.

The compounds of Examples 1 and 4 (0.3 to 30 mg/kg) produced a dose-dependant effect in the intra-plantar carrageenan induced paw oedema model of inflammation. This demonstrates that the cytokine modulatory effect seen in the LPS mouse assay translates into a functional anti-inflammatory activity.

1. A compound of formula (1) 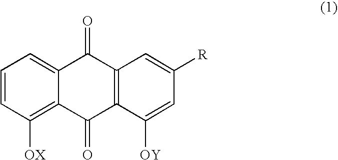
wherein
X is H or —OCR₁ and Y is H or —OCR₂, provided that X and Y are not both H; and
R is CH₂OR₉, CONR₁₁R₁₂, CN, tetrazole or COOR₁₇; wherein
R₁ and R₂ are independently selected from C₁₋₄ alkyl substituted with R₃, and a four to seven-member cyclic radical optionally substituted with a group selected from CF₃, OR₄, NR₅R₆, S(O)₀₋₂R₇, C₁₋₄ alkyl optionally substituted with R₃, and halogen, the radical also optionally containing one or more heteroatoms selected from O, S(O)₀₋₂ and NR₉;
R₃ is CF₃, OR₄, NR₅R₆ or S(O)₀₋₂R₇; or
R₄, R₅ and R₆ are independently C₁₋₄ alkyl optionally substituted with R₃, or NR₅R₆ is a C₄₋₆ heterocycloalkyl ring containing one or more additional heteroatoms selected from O, NR₉ and S(O)₀₋₂;
R₇ is C₁₋₄ alkyl;
R₉ is H or C₁₋₄ alkyl;
R₁₁ and R₁₂ are independently selected from H, OH, C₁₋₄ alkyl optionally substituted with R₁₃, C₃₋₆ cycloalkyl optionally substituted with R₁₄, and C₄₋₆ heterocycloalkyl optionally substituted with R₁₄; or NR₁₁R₁₂ is a four to seven-member cyclic radical optionally substituted with R₁₄ and optionally containing one or more heteroatoms selected from O, S(O)₀₋₂ and NR₁₅;
R₁₃ is aryl optionally substituted with R₁₄, heteroaryl optionally substituted with R₁₄, C₃₋₆ cycloalkyl optionally substituted with R₁₄, or C₄₋₆ heterocycloalkyl optionally substituted with R₁₄;
R₁₄ is OR₉, CO₂R₉ or N(R₉)₂, where each R₉ is as defined above, or N(R₉)₂ is a four to seven-member cyclic radical optionally containing one or more additional heteroatoms selected from O, S(O)₀₋₂ and NR₁₅; or N(R₉)₂ is a 5 or 6-member cyclic radical such as a lactam, succinimide or hydantoin of the formula 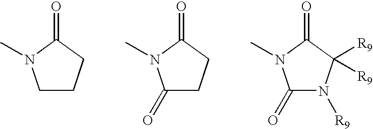
R₁₅ is R₉ or COR₁₆;
R₁₆ is C₁₋₄ alkyl, aryl or heteroaryl; and
R₁₇ is C₁₋₄ alkyl optionally substituted with R₁₃, C₃₋₆ cycloalkyl optionally substituted with R₁₄, or C₄₋₆ heterocycloalkyl optionally substituted with R₁₄;
or a salt thereof.

2. The compound according to claim 1, wherein X is —OCR₁, and Y is —OCR₂.

3. The compound according to claim 1, wherein X is —OCR₁, and Y is H.

4. The compound according to claim 1, wherein X is H and Y is —OCR₂.

5. The compound according to claim 2, wherein R₁ and R₂ are each 4-tetrahydropyranyl.

6. The compound according to claim 1, which is
1,8-bis(tetrahydropyran-4-carbonyloxy)-3-(morpholine-4-carbonyl)-9,10-dioxo-9,10-dihydroanthracene,
1,8-bis(tetrahydropyran-4-carbonyloxy)-3-ethylcarbamoyl-9,10-dioxo-9,10-dihydroanthracene,
4,5-bis(tetrahydropyran-4-carbonyloxy)-9,10-dioxo-9,10-dihydroanthracene-2-carboxylic acid, 2-methoxyethyl ester,
4,5-bis(tetrahydropyran-4-carbonyloxy)-9,10-dioxo-9,10-dihydroanthracene-2-carboxylic acid, ethyl ester,
4a,9,9a,10-tetrahydro-4,5-bis(2-(tetrahydro-2H-pyran-4-yl)-2-oxoethyl)-9,10-dioxoanthracene-2-nitrile or
4a,9,9a,10-tetrahydro-4,5-bis(2-(tetrahydro-2H-pyran-4-yl)-2-oxoethyl)-9,10-dioxoanthracene-2-(1H-tetrazol-5-yl).

7. (canceled)

8. A pharmaceutical composition comprising a compound of formula (1) 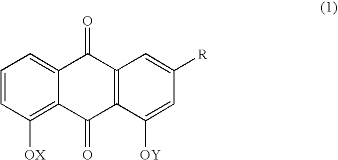
wherein
X is H or —OCR₁ and Y is H or —OCR₂, provided that X and Y are not both H; and
R is CH₂R₉, CONR₁₁R₁₂, CN, tetrazole or COOR₁₇; wherein
R₁ and R₂ are independently selected from C₁₋₄alkyl substituted with R₃, and a four to seven-member cyclic radical optionally substituted with a group selected from CF₃, OR₄, NR₅R₆, S(O)₀₋₂R₇, C₁₋₄ alkyl optionally substituted with R₃, and halogen, the radical also optionally containing one or more heteroatoms selected from O, S(O)₀₋₂ and NR₉;
R₃ is CF₃, OR₄, NR₅R₆ or S(O)₀₋₂R₇; or
R₄, R₅ and R₆ are independently C₁₋₄ alkyl optionally substituted with R₃, or NR₅R₆ is a C₄₋₆ heterocycloalkyl ring containing one or more additional heteroatoms selected from O, NR₉ and S(O)₀₋₂;
R₇ is C₁₋₄ alkyl;
R₉ is H or C₁₋₄ alkyl;
R₁₁ and R₁₂ are independently selected from H, OH, C₁₋₄ alkyl optionally substituted with R₁₃, C₃₋₆ cycloalkyl optionally substituted with R₁₄, and C₄₋₆ heterocycloalkyl optionally substituted with R₁₄; or NR₁₁R₁₂ is a four to seven-member cyclic radical optionally substituted with R₁₄ and optionally containing one or more heteroatoms selected from O, S(O)₀₋₂ and NR₁₅;
R₁₃ is aryl optionally substituted with R₁₄, heteroaryl optionally substituted with R₁₄, C₃₋₆ cycloalkyl optionally substituted with R₁₄, or C₄₋₆ heterocycloalkyl optionally substituted with R₁₄;
R₁₄ is OR₉, CO₂R₉ or N(R₉)₂, where each R₉ is as defined above, or N(R₉)₂ is a four to seven-member cyclic radical optionally containing one or more additional heteroatoms selected from O, S(O)₀₋₂ and NR₁₅; or N(R₉)₂ is a 5 or 6-member cyclic radical such as a lactam, succinimide or hydantoin of the formula 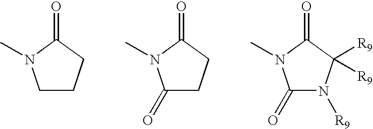
R₁₅ is R₉ or COR₁₆;
R₁₆ is C₁₋₄ alkyl, aryl or heteroaryl; and
R₁₇ is C₁₋₄ alkyl optionally substituted with R₁₃, R₃₋₆ cycloalkyl optionally substituted with R₁₄, or C₄₋₆ heterocycloalkyl optionally substituted with R₁₄;
or a salt thereof;
and a pharmaceutically acceptable diluent or carrier.

9. A method for treating a condition associated with T-cell proliferation or that is mediated by pro-inflammatory cytokines wherein said method comprises administering, to a patient in need of such treatment, a compound of formula (1) 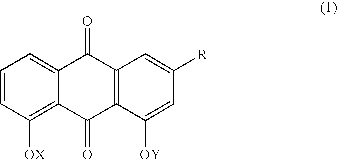
wherein
X is H or —OCR₁ and Y is H or —OCR₂, provided that X and Y are not both H; and
R is CH₂OR₉, CONR₁₁R₁₂, CN, tetrazole or COOR₁₇; wherein
R₁ and R₂ are independently selected from C₁₋₄ alkyl substituted with R₃, and a four to seven-member cyclic radical optionally substituted with a group selected from CF₃, OR₄, NR₅R₆, S(O)₀₋₂R₇, C₁₋₄ alkyl optionally substituted with R₃, and halogen, the radical also optionally containing one or more heteroatoms selected from O, S(O)₀₋₂ and NR₉;
R₃ is CF₃, OR₄, NR₅R₆ or S(O)₀₋₂R₇; or
R₄, R₅ and R₆ are independently C₁₋₄ alkyl optionally substituted with R₃, or NR₅R₆ is a C₄₋₆ heterocycloalkyl ring containing one or more additional heteroatoms selected from O, NR₉ and S(O)₀₋₂;
R₇ is C₁₋₄ alkyl;
R₉ is H or C₁₋₄ alkyl;
R₁₁ and R₁₂ are independently selected from H, OH, C₁₋₄ alkyl optionally substituted with R₁₃, C₃₋₆ cycloalkyl optionally substituted with R₁₄, and C₄₋₆ heterocycloalkyl optionally substituted with R₁₄; or NR₁₁R₁₂ is a four to seven-member cyclic radical optionally substituted with R₁₄ and optionally containing one or more heteroatoms selected from O, S(O)₀₋₂ and NR₁₅;
R₁₃ is aryl optionally substituted with R₁₄, heteroaryl optionally substituted with R₁₄, C₃₋₆ cycloalkyl optionally substituted with R₁₄, or C₄₋₆ heterocycloalkyl optionally substituted with R₁₄;
R₁₄ is OR₉, CO₂R₉ or N(R₉)₂, where each R₉ is as defined above, or N(R₉)₂ is a four to seven-member cyclic radical optionally containing one or more additional heteroatoms selected from O, S(O)₀₋₂ and NR₁₅; or N(R₉)₂ is a 5 or 6-member cyclic radical such as a lactam, succinimide or hydantoin of the formula 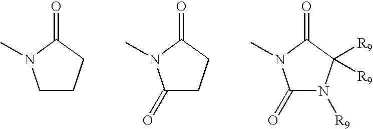
R₁₅ is R₉ or COR₁₆;
R₁₆ is C₁₋₄ alkyl, aryl or heteroaryl; and
R₁₇ is C₁₋₄ alkyl optional substituted with R₁₃, C₃₋₆ cycloalkyl optionally substituted with R₁₄ or C₄₋₆ heterocycloalkyl optionally substituted with R₁₄;
or a salt thereof.

10. The method according to claim 9, wherein the condition is a chronic degenerative disease.

11. The method according to claim 9, wherein the condition is a chronic demyelinating disease.

12. The method according to claim 9, wherein the condition is a respiratory disease.

13. The method according to claim 9, wherein the condition is an inflammatory bowel disease (IBD).

14. The method according to claim 9, wherein the condition is a dermatological condition such as psoriasis, scleroderma or atopic dermatitis.

15. The method according to claim 9, wherein the condition is a dental disease.

16. The method according to claim 9, wherein the condition is diabetic nephropathy, lupus nephritis, IgA nephropathy or glomerulonephritis.

17. The method according to claim 9, wherein the condition is systemic lupus erythematosus (SLE).

18. The method according to claim 9, wherein the condition is graft vs host disease.

19. The method according to claim 9, wherein the condition is characterised by angiogenesis.

20. The method according to claim 9, wherein the condition is cancer.

21. The method according to claim 9, wherein the condition is diabetic retinopathy or age-related macular degeneration.
